(19) United States
(12) Patent Application Publication
(10) Pub. No.: US 2024/0057909 A1
(43) Pub. Date: Feb. 22, 2024

(54) A Blood Collection Device with Improved Blood Draw Time

(71) Applicant: Becton, Dickinson and Company, Franklin Lakes, NJ (US)

(72) Inventors: Yiping Ma, Layton, UT (US); Curtis H. Blanchard, Herriman, UT (US); Jonathan Karl Burkholz, Salt Lake City, UT (US); Weston F. Harding, Lehi, UT (US)

(21) Appl. No.: 18/236,150

(22) Filed: Aug. 21, 2023

Related U.S. Application Data
(60) Provisional application No. 63/399,887, filed on Aug. 22, 2022.

(51) Int. Cl. A61B 5/15; A61B 5/153

(52) U.S. Cl. CPC A61B 5/150992; A61B 5/15003; A61B 5/153

(57) ABSTRACT

A blood collection device including a distal end portion, a proximal end portion, an actuator, and a probe operably coupled to the actuator, wherein the actuator is configured to selectively advance the probe through a catheter of a vascular access device when the blood collection device is coupled to the vascular access device. The probe includes a distal portion and a proximal portion, wherein the distal portion and the proximal portion of the probe are formed of different materials, and wherein only the distal portion of the probe is configured to extend at least partially beyond a distal tip of the catheter of the vascular access device when the probe is in a fully advanced position.

### CROSS-REFERENCE TO RELATED APPLICATION

The present application claims priority to U.S. Provisional Application Ser. No. 63/399,887, entitled “A Blood Collection Device with Improved Blood Draw Time”, filed Aug. 22, 2022, the entire disclosure of which is hereby incorporated by reference in its entirety.

### BACKGROUND OF THE INVENTION

### Field of the Invention

The present disclosure generally relates to systems and methods for collecting a blood sample from vascular access devices such as, e.g., peripheral intravenous catheters (PIVCs). More particularly, the systems described herein include blood collection devices configured to improve blood collection rate.

### Description of Related Art

Catheters are commonly used for a variety of infusion therapies. For example, catheters may be used for infusing fluids, such as normal saline solution, various medicaments, and total parenteral nutrition, into a patient. Catheters may also be used for withdrawing blood from the patient.

Blood withdrawal using a peripheral IV catheter may be difficult for several reasons, particularly when an indwelling time of the catheter is more than one day. For example, when the catheter is left inserted in the patient for a prolonged period of time, the catheter or vein may be more susceptible to narrowing, collapse, kinking, blockage by debris (e.g., fibrin or platelet clots), and adhering of a tip of the catheter to the vasculature. Due to this, catheters may often be used for acquiring a blood sample at a time of catheter placement, but are much less frequently used for acquiring a blood sample during the catheter dwell period.

Accordingly, blood collection devices have been developed to collect blood samples through an existing PIVC. These blood collection devices attach to the PIVC and include a probe or flow tube that is advanced through the PIVC, beyond the catheter tip, and into a vessel to collect a blood sample. After blood collection, the blood collection device is removed from the PIVC and discarded. One example of such a blood collection device, known as PIVO™ from Becton, Dickinson and Company, is shown and described in, e.g., U.S. Pat. No. 11,090,461, which is hereby incorporated by reference in its entirety.

FIG. 1 illustrates a blood collection system 10 including such a “line draw” blood collection device 12. The blood collection device 12 includes an introducer body 14 and an actuator 16. The actuator 16 is configured to be linearly displaceable by a clinician along a track or other feature of the introducer body 14 between a proximal end portion 18 and a distal end portion 20 of the introducer body 14. The actuator 16 is operably coupled to an elongated probe 48 to advance and retract the probe 48 through a distal core portion (not shown) located near the distal end portion 20 of the introducer body 14, thereby allowing the probe 48 to enter the patient's vasculature via an indwelling catheter 46 when the blood collection device 12 is coupled to a catheter adapter 44.

A connector member 22 is configured to selectively couple the blood collection device 12 to, e.g., a needleless access connector 42 extending from the catheter adapter 44. A proximal extension tube 26 extends from the proximal end portion 18 of the introducer body 14, with the proximal extension tube 26 being fluidly coupled to the probe 48 of the blood collection device 12. An occlusion device 28 such as, e.g., a clamp may be provided in-line with the proximal extension tube 26 to enable a user to selectively block fluid flow through the proximal extension tube 26. A proximal connector 30 may be provided on a proximal end of the proximal extension tube 26 such that the blood draw system 10 may be coupled to an appropriate blood collection interface such as, e.g., a luer lock access device, thereby enabling blood collection via the blood collection device 12.

While blood collection devices such as blood collection device 12 succeed in allowing blood collection via a catheter over a longer indwell period, the rate of blood collection with such devices is often quite slow, as the blood flow path is confined within the elongated probe 48 and the proximal extension tube 26. As the probe 48 is configured to extend beyond the distal tip of the catheter 46, the diameter of the probe 48 must be smaller than the inside diameter of the distal tip of the catheter 46, thereby limiting the blood collection rate, which is particularly noticeable when used with small gauge (e.g., 24G) catheters.

### SUMMARY OF THE INVENTION

In accordance with an aspect of the present disclosure, a blood collection device includes a distal end portion, a proximal end portion, an actuator, and a probe operably coupled to the actuator. The actuator is configured to selectively advance the probe through a catheter of a vascular access device when the blood collection device is coupled to the vascular access device. The probe includes a distal portion and a proximal portion, wherein the distal portion and the proximal portion of the probe are formed of different materials, and wherein only the distal portion of the probe is configured to extend at least partially beyond a distal tip of the catheter of the vascular access device when the probe is in a fully advanced position.

In certain embodiments, the distal portion of the probe comprises a tubular section and the proximal portion of the probe comprises a wire section. The proximal portion of the probe further may include at least one coil section. The distal portion of the probe may further include a distal coil positioned at a distal end of the tubular section. The distal end of the tubular section may be capped.

In accordance with certain embodiments, the tubular section includes at least one perforation formed in a sidewall thereof. The wire section may be coupled to a proximal end portion of the tubular section via an adhesive. The tubular section and the wire section may be co-extruded.

In accordance with certain embodiments, the distal portion of the probe includes a wire coil and the proximal portion of the probe comprises a solid, flexible rod. The wire coil may include a variable pitch wire coil. The wire coil may include an atraumatic tip. The distal portion of the probe may include a Nitinol tubular section.

In accordance with certain embodiments, the proximal portion of the probe may include a polyurethane tubular section. An inner diameter of the Nitinol tubular section may be smaller than an inner diameter of the polyurethane tubular section. The Nitinol tubular section and the polyurethane tubular section may be coupled by a shrink joint.

In accordance with another embodiment of the present invention a method of blood sample collection from a patient includes providing a vascular access device comprising a catheter adapter and a catheter, inserting the catheter of the vascular access device into a vein of the patient, and providing a blood collection device. The blood collection device includes a distal end portion, a proximal end portion, an actuator, and a probe operably coupled to the actuator. The probe includes a distal portion and a proximal portion, and the distal portion and the proximal portion of the probe are formed of different materials. The method also includes coupling the blood collection device to the vascular access device, advancing the probe of the blood collection device through the vascular access device such that only the distal portion of the probe extends beyond a distal tip of the catheter, and drawing blood through the distal portion of the probe and into the catheter.

In certain embodiments, the probe is disposed at least partially within a tubular tube, and the tubular tube spaced circumferentially apart from the probe. The tubular tube 104 and the probe 106 may be configured to be selectively advanced through the vascular access device. The method may also include drawing blood into a blood collection container. The blood collection container may be fluidly coupled to the blood collection device. The blood collection container may be fluidly coupled to the catheter adapter. The method may also include advancing the probe through a first port in the catheter adapter and drawing a blood sample through a second port in the catheter adapter.

Further details and advantages of the invention will become clear upon reading the following detailed description in conjunction with the accompanying drawing figures, wherein like parts are designated with like reference numerals throughout.

### BRIEF DESCRIPTION OF THE DRAWINGS

FIG. 1 is a plan view of a blood collection system in accordance with the prior art;

FIG. 2A is a partial side cross-sectional view of a probe and catheter in accordance with an aspect of the present disclosure;

FIG. 2B is a partial side view the probe of FIG. 2A;

FIG. 2C is a partial cross-sectional view of the probe of FIG. 2A;

FIG. 3A is a partial side view of a probe in accordance with another aspect of the present disclosure;

FIG. 3B is a partial side view the probe of FIG. 3A and a cross-sectional view of a catheter in accordance with the present disclosure;

FIG. 3C is a partial side view of the probe of FIG. 3A in a first configuration;

FIG. 3D is a partial side view of the probe of FIG. 3A in a second configuration;

FIG. 3E is a cross-sectional end view of the probe of FIG. 3A;

FIG. 4 is a partial side view of a probe and catheter in accordance with another aspect of the present disclosure;

FIG. 4A is an enlarged portion view of FIG. 4 denoted by section 4A;

FIG. 5 is a partial side view of a probe in accordance with another aspect of the present disclosure; and

FIG. 6 is a partial cross-sectional view of a vascular access device in accordance with another aspect of the present disclosure.

### DESCRIPTION OF PREFERRED EMBODIMENTS

The following description is provided to enable those skilled in the art to make and use the described aspects contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present disclosure.

For the purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawings. However, it is to be understood that the invention may assume various alternative variations, except where expressly specified to the contrary. It is also to be understood that the specific devices illustrated in the attached drawings, and described in the following specification, are simply exemplary aspects of the invention. Hence, specific dimensions and other physical characteristics related to the aspects disclosed herein are not to be considered as limiting.

In the present disclosure, the distal end of a component or of a device means the end furthest away from the hand of the user and the proximal end means the end closest to the hand of the user, when the component or device is in the use position, i.e., when the user is holding a blood collection device in preparation for or during use. Similarly, in this application, the terms “in the distal direction” and “distally” mean in the direction toward an access connector portion of the blood collection device, and the terms “in the proximal direction” and “proximally” mean in the direction opposite the direction of the connector.

While not shown or described herein, it is to be understood that the blood collection systems described below may be utilized for blood collection from any suitable vascular access device such as, e.g., the BD NEXIVA™ Closed IV Catheter system, the BD CATHENA™ Catheter system, the BD VENFLON™ Pro Safely Shielded IV Catheter system, the BD NEOFLON™ IV Cannula system, the BD INSYTE™ AUTOGUARD™ BC Shielded IV Catheter system, or another suitable vascular access device.

Embodiments of the present disclosure will primarily be described in the context of blood sample collection systems for use with PIVCs. However, embodiments of the present disclosure equally extend to use with other catheter devices.

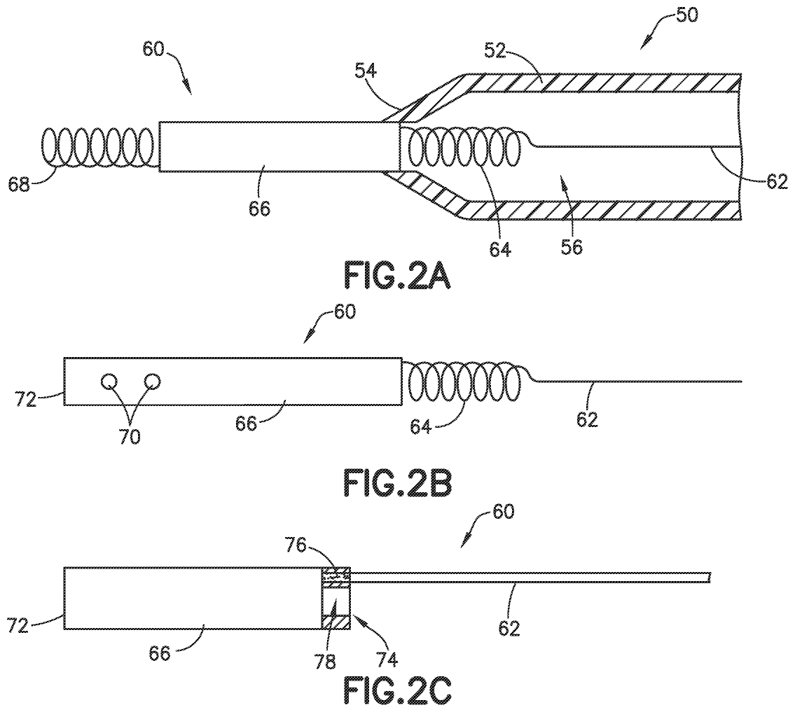

Referring to FIGS. 2A-2C, a partial view of a blood collection system 50 in accordance with an aspect of the present disclosure is illustrated. While not shown, it is to be understood that the blood collection system 50 may include a blood collection device such as, e.g., a PIVO™ device from Becton, Dickinson and Company, as well as a vascular access device such as, e.g., the BD NEXIVA™ Closed IV Catheter system, the BD CATHENA™ Catheter system, the BD VENFLON™ Pro Safely Shielded IV Catheter system, the BD NEOFLON™ IV Cannula system, the BD INSYTE™ AUTOGUARD™ BC Shielded IV Catheter system, or another suitable vascular access device.

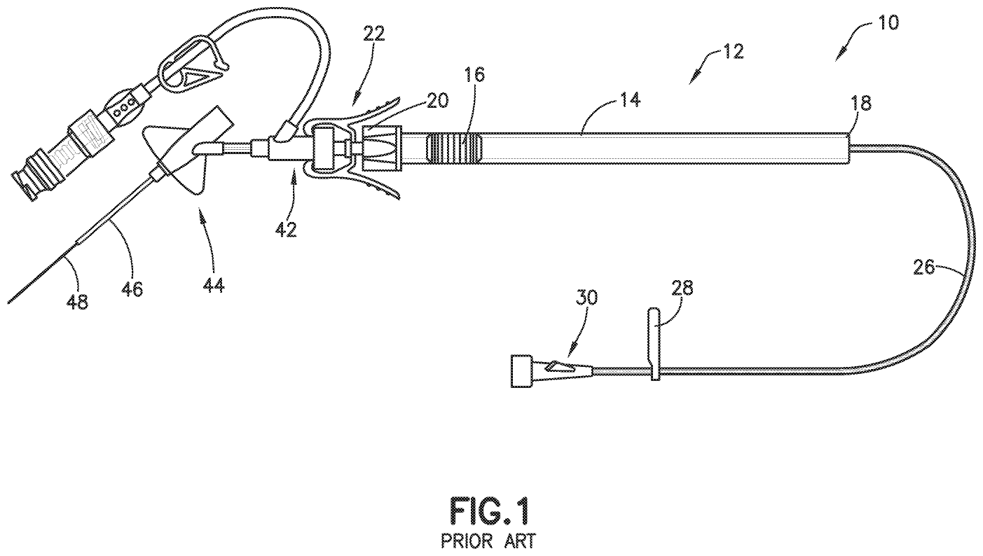

Blood collection system 50 includes a catheter 52 having a tapered distal end 54 and an inner conduit 56 formed therethrough. Similar to catheter 46 described above with respect to FIG. 1, catheter 52 may extend from a catheter adapter of a vascular access system to provide access to a patient's vasculature when indwelled. The catheter 52 may be formed of any appropriate material and may be any appropriate length and gauge such as, e.g., 18G, 20G, 22G, 24G, etc.

Additionally, blood collection system 50 includes a probe 60. While not shown, it is to be understood that probe 60 is configured to be selectively advanced through the catheter 52 by a blood collection device such as, e.g., a PIVO™ device from Becton, Dickinson and Company. However, unlike probe 48 described above with respect to FIG. 1, which has a substantially constant inner diameter along its entire length, the probe 60 includes two or more separate sections, with the sections configured to increase blood collection rate by minimizing the tubular section of probe 60 which extends beyond the tapered distal end 54 of the catheter 52 in a fully advanced position.

Specifically, in the embodiment shown in FIGS. 2A-2C, probe 60 includes a proximal wire section 62 and a distal tubular section 66. While not shown, it is to be understood that proximal wire section 62 is configured to be coupled to an actuator of a blood collection device so as to allow for advancement of the probe 60 through the catheter 52. In some embodiments, the proximal wire section 62 may include one or more coil sections 64, and may comprise any combination of wires and coils.

FIG. 2A shows the probe 60 in its distal-most position when advanced through the catheter 52, wherein the distal tubular section 66 extends beyond the distal end 54 of the catheter 52 and into a vein of the patient, while the proximal wire section 62 remains within the inner conduit 56 of the catheter 52. In this way, blood may be drawn into (or otherwise enter) the distal tubular section 66 for collection into an appropriate container coupled to the blood collection device, with the distal tubular section 66 having an outer diameter smaller than the inner diameter of the distal end 54 of the catheter 52 so as to allow for passage of the probe 60 therethrough. However, because the length of the distal tubular section 66 only extends to the distal end 54 of the catheter 52 in the fully-advanced position of probe 60, the flow restriction caused by the relatively small inner diameter of the distal tubular section 66 may be minimized. Instead, blood flowing from the distal tubular section 66 exits the distal tubular section 66 slightly proximal the distal end 54, thereby allowing the blood sample to flow around the proximal wire section 62 and through the inner conduit 56 of the catheter 52 and to the blood collection device. As the inner conduit 56 has an inner diameter that is notably larger than the inner diameter of the distal tubular section 66, the collection rate of the blood sample may be increased as compared to the prior art probe devices.

Referring still to FIG. 2A, in some embodiments, the distal tubular section 66 may include a distal coil 68, wherein the distal coil 68 may be capped so as to provide an atraumatic tip to the probe 60 to aid in the prevention of damage to the inner walls of the patient's vein as the probe 60 is advanced from the catheter 50. Additionally, capping the distal end of the distal coil 68 may also prevent thrombus entrapment during deployment of the probe 60. Referring to FIG. 2B, in other embodiments, the distal tubular section 66 may include one or more perforations 70 formed through a sidewall thereof, with a distal cap 72 being provided to close a distal end of the distal tubular section 66. The perforations 70 may be configured to provide an inlet for blood into the probe 60.

Referring to FIG. 2C, in some embodiments, the proximal wire section 62 may be coupled to a proximal end portion 74 of the distal tubular section 66, with only a distal end portion 76 of the proximal wire section 62 being retained via over-molding with the distal tubular section 66. In some embodiments, the distal end portion 76 of the proximal wire section 62 may be held within the distal tubular section 66 via an adhesive such as, e.g., glue. As shown in FIG. 2C, proximal end portion 74 may include an opening 78 formed so as to allow blood to flow therethrough, with the proximal wire section 62 offset from the opening 78.

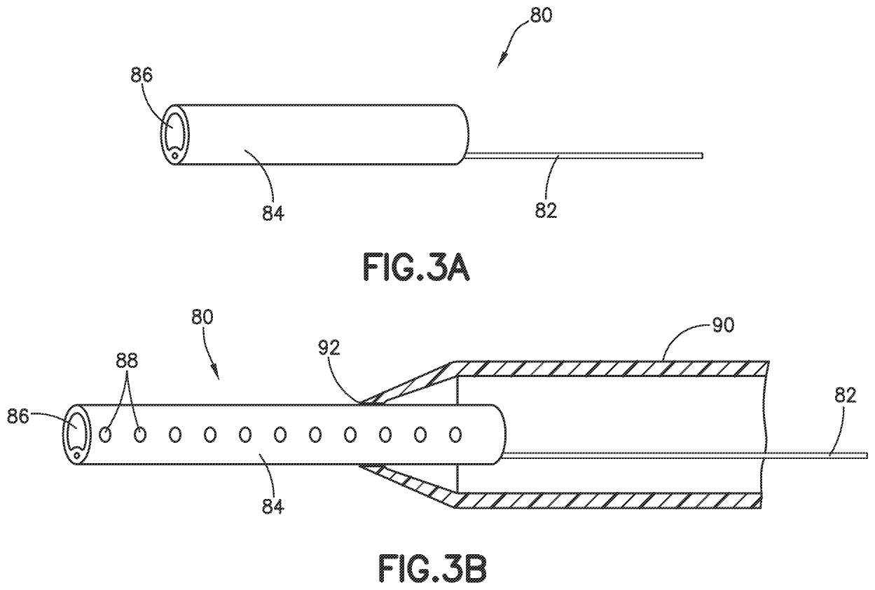

Next, referring to FIGS. 3A-3E, a probe 80 in accordance with another aspect of the present disclosure is shown. Unlike probe 60 described above with respect to FIGS. 2A-2C, which included only a small distal portion of the proximal wire section 62 over-molded by the distal tubular section 66 and/or retained with an adhesive, the probe 80 is initially formed such that an advancement wire 82 is co-extruded with a tubular section 84. Then, as shown in FIG. 3C, a proximal length 85 of the tubular section 84 can be stripped from the advancement wire 82, resulting in a proximal section of the advancement wire 82 extending from the tubular section 84, as is shown in FIGS. 3A and 3D.

In some embodiments, the tubular section 84 may include a distal opening 86 to allow blood to flow therein. However, in other embodiments, the distal end of the tubular section 84 may be capped and/or may be configured to provide an atraumatic tip. As shown in FIG. 3B, in some embodiments, the tubular section 84 may include a plurality of side openings 88 to allow blood to flow into the tubular section 84 for collection in a blood collection container (not shown).

FIG. 3B shows the probe 80 in a distally advanced position, with at least a portion of the tubular section 84 extending beyond a distal tip 92 of a catheter 90, while the advancement wire 82 remains within the catheter 90. In this way, blood may be drawn into (or otherwise enter) the tubular section 84 for collection into an appropriate container coupled to the blood collection device. However, because the length of the tubular section 84 only extends to the distal tip 92 of the catheter 90 in the fully-advanced position of probe 80, the flow restriction caused by the relatively small inner diameter of the tubular section 84 may be minimized, thereby allowing the blood sample to flow around the advancement wire 82, through an inner conduit of the catheter 90, and to the blood collection device, which results in an increased flow rate of the blood sample as compared to the prior art probe devices.

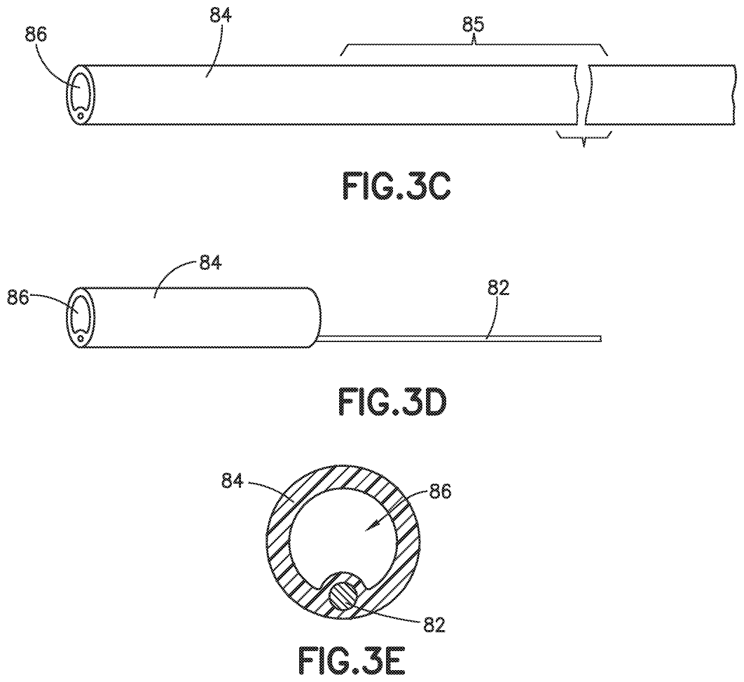

Next, referring to FIG. 4, a blood collection system 100 in accordance with another aspect of the present disclosure is shown. While not shown, it is to be understood that the blood collection system 100 may include a blood collection device such as, e.g., a PIVO™ device from Becton, Dickinson and Company, as well as a vascular access device such as, e.g., the BD NEXIVA™ Closed IV Catheter system, the BD CATHENA™ Catheter system, the BD VENFLON™ Pro Safely Shielded IV Catheter system, the BD NEOFLON™ IV Cannula system, the BD INSYTE™ AUTOGUARD™ BC Shielded IV Catheter system, or another suitable vascular access device.

Blood collection system 100 includes a catheter 108 having a tapered distal end 109, with the catheter 108 extending from a catheter adapter 102 of the vascular access device. While not shown, it is to be understood that the catheter adapter 102 may be coupled to, e.g., an integrated extension set.

Additionally, blood collection system 100 includes a probe 106 inside a tubular tube 104, such that the tubular tube 104 may be circumferentially spaced from the probe 106. While not shown, it is to be understood that probe 106 along with the tubular tube 104 is configured to be selectively advanced through the catheter adapter 102 and at least partially through the catheter 108 by a blood collection device such as, e.g., a PIVO™ device from Becton, Dickinson and Company. As shown in FIG. 4A, blood may flow in the interior space (as shown by flow directional indicators 103), around the probe 106 and within the tubular tube 104. The probe 106 may be configured as, e.g., a solid, flexible rod formed of any appropriate material.

Referring still to FIG. 4, a wire coil 112 is coupled to a distal end portion 110 of the probe 106. In some embodiments, the wire coil 112 is a variable pitch wire coil. Accordingly, when the probe 106 is advanced toward its distal-most position through the catheter 108, only the wire coil 112 extends beyond the distal end 109 of the catheter 108 and into a vein of the patient, while the solid, flexible rod portion of the probe 106 remains within the inner conduit of the catheter 108. In this way, the fluid-permeable wire coil 112 facilitates blood collection into the catheter 108 in the presence of thrombus around the distal end 109 of catheter 108, avoiding the high fluid resistance of prior art probes formed as an elongated tube. Additionally, in some embodiments, the tubular tube 104 extended only within the catheter adapter 102 in the fully advanced position may further act to minimize potential sample contamination from, e.g., any residual medication that was not previously flushed from within the catheter adapter 102 prior to a blood collection procedure.

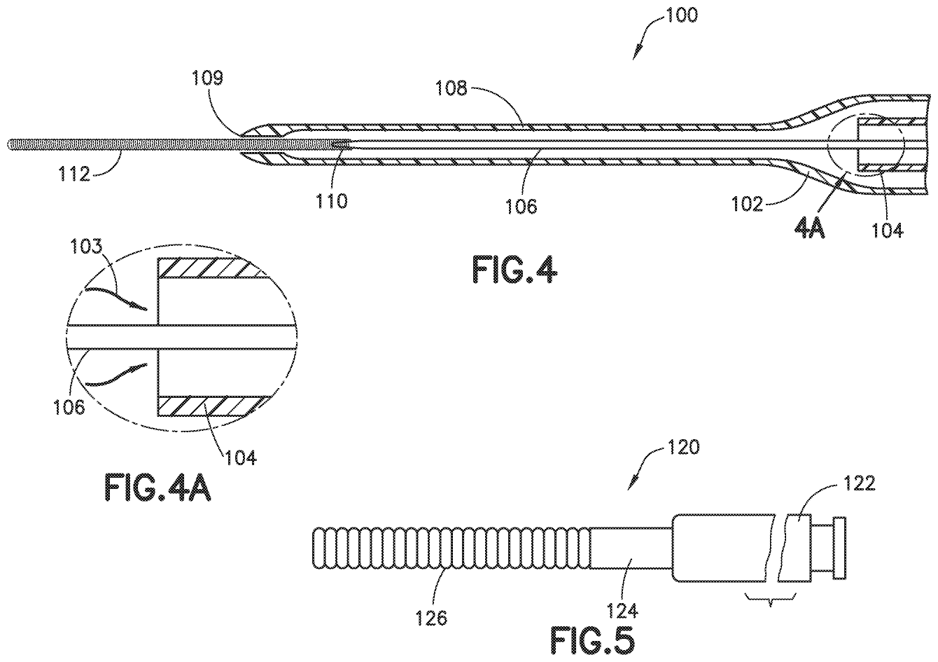

Next, referring to FIG. 5, a probe 120 in accordance with another aspect of the present disclosure is illustrated. While not shown, it is to be understood that probe 120 may be configured to be selectively advanced through a vascular access device by a blood collection device such as, e.g., a PIVO™ device from Becton, Dickinson and Company. The probe 120 includes a proximal tubular section 122 formed of, e.g., polyurethane, a Nitinol distal tubular section 126, and a shrink joint 124 coupling the proximal tubular section 122 and the Nitinol distal tubular section 126. Nitinol is a nickel and titanium alloy having high elasticity and “shape memory” properties. Accordingly, the Nitinol distal tubular section 126 may be formed with a thinner wall than conventional probes, thereby increasing the inner diameter and, thus, the cross-sectional area of the fluid path at the distal portion of the probe 120, which is typically the most fluid-restrictive portion of a probe. With such an increased cross-sectional area at the distal portion of the probe 120, flow rate through the probe 120 may be increased. Alternatively, in other embodiments, the Nitinol distal tubular section 126 could be replaced with a polyimide tubing.

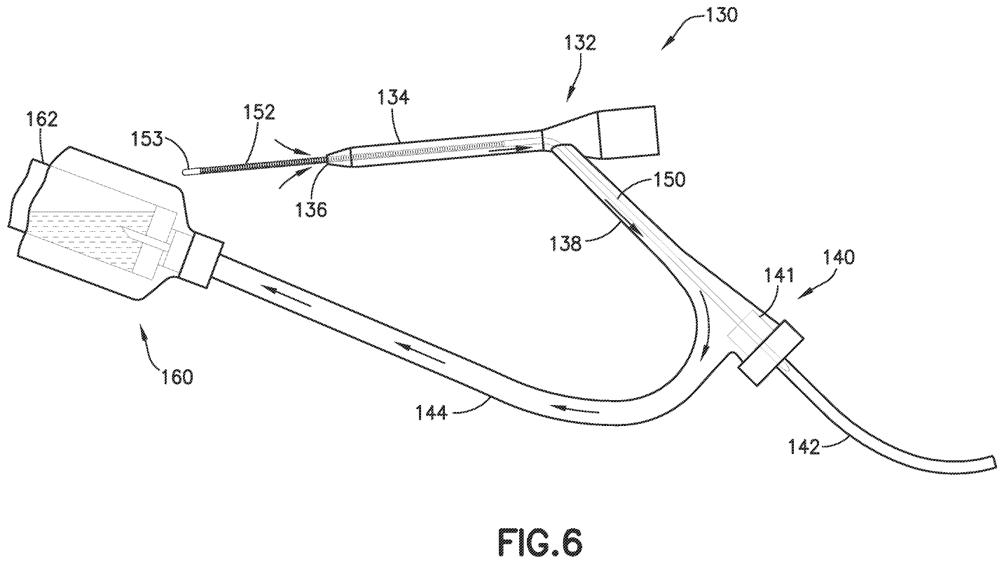

Referring now to FIG. 6, a blood collection system 130 in accordance with another aspect of the present disclosure is shown. Blood collection system 130 includes a vascular access device 132 having a catheter 134 extending therefrom, with the catheter 134 having a distal tip 136. An adapter 140 is coupled to the vascular access device 132. In some embodiments, the adapter 140 may be coupled to the vascular access device 132 by, e.g., a length of extension tubing 138. However, it is to be understood that adapter 140 may be integrated with the vascular access device 132.

As shown in FIG. 6, the adapter 140 may be provided with at least two access ports, wherein a first access port may be coupled to a first extension tube 142 and the second access port may be coupled a second extension tube 144. The first extension tube 142 may provide a non-fluid collection path, while the second extension tube 144 may be configured for fluid collection, more specifically blood sample collection. In some embodiments, the second extension tube 144 may be coupled to a blood collection interface such as, e.g., a luer lock access device 160, which may be configured to receive a blood collection container 162 therein for the collection of one or more blood samples.

Blood collection system 130 may further include a probe 150, with the probe 150 configured to extend through the extension tubing 138 and at least a portion of the catheter 134 when in a fully advanced position. In the embodiment shown in FIG. 6, the probe 150 is a solid, flexible rod, with a proximal end of the probe 150 configured to pass through a male luer connector 141 of the extension tubing 138. In some embodiments, the probe 150 may be advanced through the adapter 140 and the catheter 134 by a blood collection device such as, e.g., a PIVO™ device from Becton, Dickinson and Company. However, in other embodiments, the probe 150 may be advanced and positioned within the vascular access device 132 by another, alternative means.

Referring still to FIG. 6, a wire coil 152 is coupled to a distal end portion of the probe 150. In some embodiments, the wire coil 152 is a variable pitch wire coil. In some embodiments, the wire coil 152 may include an atraumatic tip 153. When the probe 150 is advanced or otherwise deployed toward its distal-most position through the catheter 134, only the wire coil 152 extends beyond the distal end 136 of the catheter 134 and into a vein of the patient, while the solid, flexible rod portion of the probe 150 remains within the inner conduit of the catheter 134. In this way, the fluid-permeable wire coil 152 facilitates blood collection into the catheter 134 in the presence of thrombus around the distal end 136 of catheter 134, avoiding the high fluid resistance of prior art probes formed as an elongated tube, and thereby providing for increased blood collection rate as compared to prior art probe concepts.

As shown in FIG. 6, the adapter 140 is configured such that the probe 150 (with wire coil 152) is inserted through the first port, while blood is drawn or otherwise received through the second port. Such a two-port configuration, coupled with the low fluid resistance of the fluid-permeable wire coil 152, enables blood to be drawn into the blood collection container 162 at an increased flow rate.

While several embodiments of blood collection systems configured for blood collection from an indwelling catheter were described in the foregoing detailed description, those skilled in the art may make modifications and alterations to these embodiments without departing from the scope and spirit of the invention. Accordingly, the foregoing description is intended to be illustrative rather than restrictive. The invention described hereinabove is defined by the appended claims and all changes to the invention that fall within the meaning and the range of equivalency of the claims are embraced within their scope.

What is claimed is:

1. A blood collection device comprising:
a distal end portion;
a proximal end portion;
an actuator; and
a probe operably coupled to the actuator,
wherein the actuator is configured to selectively advance the probe through a catheter of a vascular access device when the blood collection device is coupled to the vascular access device,
wherein the probe comprises a distal portion and a proximal portion,
wherein the distal portion and the proximal portion of the probe are formed of different materials, and
wherein only the distal portion of the probe is configured to extend at least partially beyond a distal tip of the catheter of the vascular access device when the probe is in a fully advanced position.

2. The blood collection device of claim 1, wherein the distal portion of the probe comprises a tubular section and the proximal portion of the probe comprises a wire section.

3. The blood collection device of claim 2, wherein the proximal portion of the probe further comprises at least one coil section.

4. The blood collection device of claim 2, wherein the distal portion of the probe further comprises a distal coil positioned at a distal end of the tubular section.

5. The blood collection device of claim 2, wherein a distal end of the tubular section is capped.

6. The blood collection of claim 2, wherein the tubular section comprises at least one perforation formed in a sidewall thereof.

7. The blood collection device of claim 2, wherein the wire section is coupled to a proximal end portion of the tubular section via an adhesive.

8. The blood collection device of claim 2, wherein the tubular section and the wire section are co-extruded.

9. The blood collection device of claim 1, wherein the distal portion of the probe comprises a wire coil and the proximal portion of the probe comprises a solid, flexible rod.

10. The blood collection device of claim 9, wherein the wire coil comprises a variable pitch wire coil.

11. The blood collection device of claim 9, wherein the wire coil comprises an atraumatic tip.

12. The blood collection device of claim 1, wherein the distal portion of the probe comprises a Nitinol tubular section.

13. The blood collection device of claim 12, wherein the proximal portion of the probe comprises a polyurethane tubular section.

14. The blood collection device of claim 13, wherein an inner diameter of the Nitinol tubular section is smaller than an inner diameter of the polyurethane tubular section.

15. The blood collection device of claim 13, wherein the Nitinol tubular section and the polyurethane tubular section are coupled by a shrink joint.

16. A method of blood sample collection from a patient, the method comprising:
providing a vascular access device comprising a catheter adapter and a catheter;
inserting the catheter of the vascular access device into a vein of the patient;
providing a blood collection device, the blood collection device comprising:
a distal end portion;
a proximal end portion;
an actuator; and
a probe operably coupled to the actuator,
wherein the probe comprises a distal portion and a proximal portion, and
wherein the distal portion and the proximal portion of the probe are formed of different materials;
coupling the blood collection device to the vascular access device;
advancing the probe of the blood collection device through the vascular access device such that only the distal portion of the probe extends beyond a distal tip of the catheter; and
drawing blood through the distal portion of the probe and into the catheter.

17. The method of claim 16, wherein the probe is disposed at least partially within a tubular tube, and the tubular tube spaced circumferentially apart from the probe.

18. The method of claim 17, wherein the tubular tube and the probe are configured to be selectively advanced through the vascular access device.

19. The method of claim 16, further comprising drawing blood into a blood collection container.

20. The method of claim 19, wherein the blood collection container is fluidly coupled to the blood collection device.

21. The method of claim 19, wherein the blood collection container is fluidly coupled to the catheter adapter.

22. The method of claim 16, further comprising advancing the probe through a first port in the catheter adapter and drawing a blood sample through a second port in the catheter adapter.
